(19) United States
(12) Patent Application Publication
(10) Pub. No.: US 2003/0139424 A1
(43) Pub. Date: Jul. 24, 2003

(54) Use

(76) Inventor: Patrizia Caldirola, Uppsala (SE)

Correspondence Address: FISH & RICHARDSON PC, 225 FRANKLIN ST, BOSTON, MA, 02110, US

(21) Appl. No.: 10/277,299

(22) Filed: Oct. 22, 2002

Related U.S. Application Data
(60) Provisional application No. 60/340,599, filed on Dec. 14, 2001.

(30) Foreign Application Priority Data
Oct. 23, 2001 (SE) 0103539-3

(51) Int. Cl. A61K 31/5377; A61K 31/496; A61K 31/454; A61K 31/405

(52) U.S. Cl. 514/254.09; 514/232.8; 514/414; 514/419; 514/323

(57) ABSTRACT

The invention provides a method of treatment or prophylaxis of obesity or for the reduction of food intake, comprising administering to a patient in need of such treatment a therapeutically effective amount of an indole or indoline derivative of Formula I, II or III:

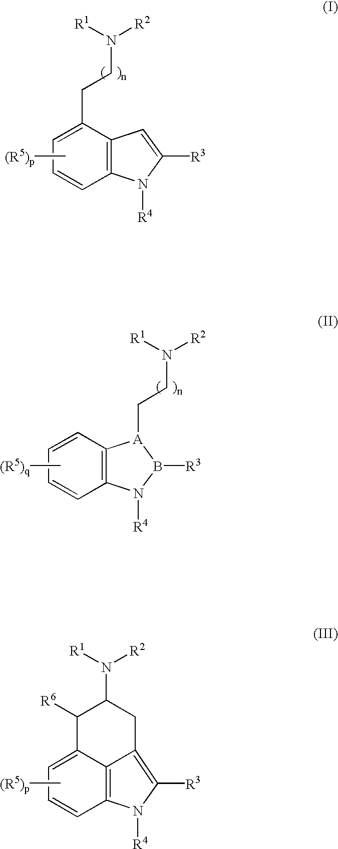

wherein the substituents are as described in the specification.

### CROSS-REFERENCE TO RELATED APPLICATIONS

This application claims benefit of Swedish Patent Application No. 0103539-3, filed Oct. 23, 2001, and U.S. Provisional Patent Application Serial No. 60/340,599, filed Dec. 14, 2001. These applications are incorporated herein by reference in their entirety.

### TECHNICAL FIELD

The present invention relates to the use of indole and indoline derivatives, which bind selectively to 5-HT₆ receptors, in the treatment of obesity or for the reduction of food intake.

### BACKGROUND ART

Obesity is a condition characterized in an increase in body fat content resulting in excess body weight above accepted norms. Obesity is the most important nutritional disorder in the western world and represents a major health problem in all industrialized countries. This disorder leads to increased mortality due to increased incidences of diseases such as cardiovascular disease, digestive disease, respiratory disease, cancer and NIDDM (type II diabetes). Searching for compounds which reduce body weight has been going on for many decades. One line of research has been activation of serotonergic systems, either by direct activation of serotonin receptor subtypes or by inhibiting serotonin re-uptake. The exact receptor subtype profile required is however not known.

Serotonin (5-hydroxytryptamine or 5-HT), a key transmitter of the peripheral and central nervous system, modulate a wide range of physiological and pathological functions, including anxiety, sleep regulation, aggression, feeding and depression. Multiple serotonin receptor subtypes have been identified and cloned. One of these, the 5-HT₆ receptor, was cloned by several groups in 1993 (Ruat et al. (1993) Biochem. Biophys. Res. Commun., 193: 268-276; Sebben et al. (1994) NeuroReport 5: 2553-2557) This receptor is positively coupled to adenylyl cyclase and displays affinity for antidepressants such as clozapine. Recently, the effect of 5-HT₆ antagonist and 5-HT₆ antisense oligonucleotides to reduce food intake in rats has been reported (Bentley et al. (1999) Br. J. Pharmac. Suppl 126: P66; Bentley et al. (1997) J. Psychopharmacol. Suppl. A64: 255; Woolley, M. L. et al. (2001) Neuropharmacology 41: 210-219).

U.S. Pat. No. 6,187,805 (see also Russell, M. G. N. et al. (2001) “N-Arylsulfonylindole Derivatives as Serotonin 5-HT₆ Receptor Ligands”, J. Med. Chem. in press) disclose indole and indoline derivatives as ligands selective for the 5-HT₆ receptors, and of proposed value in the treatment or prevention of CNS disorders, including Alzheimer's disease, Parkinson's disease, schizophrenia, depression and anxiety. However, such compounds have not been disclosed that such derivatives are useful for the treatment of obesity.

### BRIEF DESCRIPTION OF THE DRAWINGS

FIG. 1 is a graph depicting the effect on food intake in obese mice by administration of a compound according to the invention.

### DISCLOSURE OF THE INVENTION

It has been found that 5-HT₆ receptor antagonists, belonging to the class of indole or indoline derivatives disclosed in U.S. Pat. No. 6,187,805, reduce food intake and body weight. Consequently, the present invention provides a method for the treatment or prophylaxis of obesity or for the reduction of food intake in mammals, including humans. The method comprises administering to a patient in need of such treatment a therapeutically effective amount of a compound of formula I, II or III:

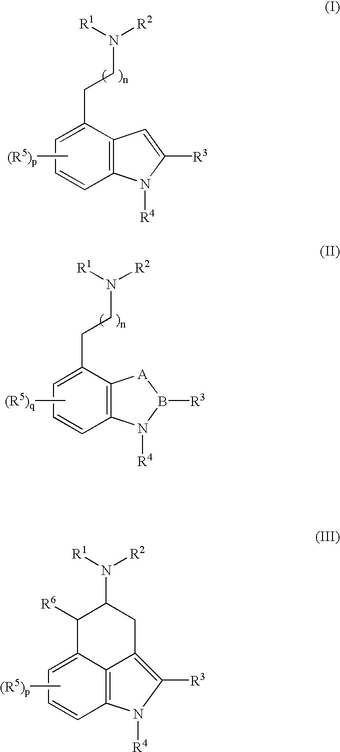

wherein

n is 1 or 2;

p is 0, 1, 2 or 3;

q is 0, 1, 2, 3 or 4;

R¹ and R² independently represent hydrogen, C₁₋₆ alkyl or aryl (C₁₋₆)alkyl, or together represent the atoms necessary to complete a heterocycloalkyl group comprising the nitrogen atom to which R¹ and R² are attached;

R³ represents hydrogen, C₁₋₆ alkyl, C₂₋₆ alkenyl, C₂₋₆ alkynyl, aryl(C₁₋₆)alkyl, aryl, heteroaryl, arylcarbonyl, heteroarylcarbonyl, C₁₋₆ alkylcarbonyl, or C₁₋₆ alkoxycarbonyl;

R⁴ represents arylsulphonyl, heteroarylsulphonyl, C₁₋₆ alkylsulphonyl, di(C₁₋₆)alkylaminosulphonyl, arylcarbonyl, C₁₋₆ alkylcarbonyl, heteroarylcarbonyl or C₁₋₆ alkoxycarbonyl;

each R⁵ independently represents hydrogen, hydroxy, C₁₋₆ alkoxy, aryl(C₁₋₆)alkoxy, nitrile or halogen;

R⁶ represents hydrogen, hydroxy or C₁₋₆ alkoxy; and

A—B represents C═C or CH═CH.

In Formulae I-III, one or more substituents may be present on any alkyl or aryl group represented by any of R¹-R⁵, or on any alkyl or aryl moiety of a group represented by any of R¹-R⁵. Preferred substituents include C₁₋₆ alkyl, halogen, hydroxy and C₁₋₆ alkoxy.

The expression “C₁₋₆alkyl” includes methyl and ethyl groups, and straight-chained, branched or cyclic propyl, butyl, pentyl and hexyl groups. Particular alkyl groups are methyl, ethyl, n-propyl, isopropyl and tert-butyl. Derived expressions such as “C₁₋₆ alkoxy”, “C₁₋₆ alkylthio” and “C₁₋₆ alkylamino” are to be construed accordingly.

The expression “C₂₋₆ alkenyl” as used herein refers to straight-chained and branched alkenyl groups containing from 2 to 6 carbon atoms. Typical examples include vinyl, allyl, dimethylallyl and butenyl groups.

The expression “C₂₋₆ alkynyl” as used herein refers to straight-chained and branched alkynyl groups containing from 2 to 6 carbon atoms. Typical examples include ethynyl and propargyl groups.

The term “aryl” refers to an aromatic ring system (monocyclic or bicyclic, only one ring need be aromatic) having from 6 to 10 ring carbon atoms. Typical aryl groups include phenyl and naphthyl.

The expression “aryl(C₁₋₆)alkyl” as used herein includes benzyl, phenylethyl, phenylpropyl and naphthylmethyl.

Suitable heterocycloalkyl groups include azetidinyl, pyrrolidinyl, piperidinyl, piperazinyl and morpholinyl groups.

The term “heteroaryl” refers to an aromatic ring system (monocyclic or bicyclic, only one ring need be aromatic) having from 5 to 10 ring atoms, in which one or more of the rings atoms are heteroatoms, such as nitrogen, sulphur, and oxygen, and the remainder are carbon atoms. Suitable heteroaryl groups include pyridinyl, quinolinyl, isoquinolinyl, pyridazinyl, pyrimidinyl, pyrazinyl, furyl, benzofuryl, dibenzofuryl, thienyl, benzthienyl, pyrrolyl, indolyl, pyrazolyl, indazolyl, oxazolyl, isoxazolyl, thiazolyl, isothiazolyl, imidazolyl, benzimidazolyl, oxadiazolyl, thiadiazolyl, triazolyl and tetrazolyl groups.

The term “halogen” as used herein includes fluorine, chlorine, bromine and iodine, especially chlorine or fluorine.

For use in medicine, the salts of the compounds of Formulae I-III will be pharmaceutically acceptable salts. Other salts may, however, be useful in the preparation of the compounds of Formulae I-III or of their pharmaceutically acceptable salts. Suitable pharmaceutically acceptable salts of the compounds of Formulae I-III include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Furthermore, where the compounds of Formulae I-III carry an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

The present invention includes within its scope the use of prodrugs of the compounds of Formulae I-III above. In general, such prodrugs will be functional derivatives of the compounds of Formulae I-III which are readily convertible in vivo into the required compounds of Formulae I-III. Conventional procedures for the selection and preparation of suitable prodrug derivatives are described, for example, in Design of Prodrugs, ed. H. Bundgaard, Elsevier, 1985.

Certain compounds according to the present invention may be capable of existing as tautomeric forms. It is to be understood that all possible tautomers and mixtures thereof in any proportion are encompassed within the scope of the present invention.

Where the compounds according to the invention have at least one asymmetric center, they may accordingly exist as enantiomers. Where the compounds according to the invention possess two or more asymmetric centers, they may additionally exist as diastereoisomers. It is to be understood that all such isomers and mixtures thereof in any proportion are encompassed within the scope of the present invention.

In Formulae I-III, suitable separate identities for R¹ and R² include hydrogen, methyl, ethyl, propyl and benzyl, and suitable identities for R¹ and R² in combination include pyrrolidinyl, piperidinyl, piperazinyl, 4-methylpiperazinyl and morpholinyl.

Suitable identities for R³ include hydrogen, methyl, ethyl, benzyl, allyl, propargyl, benzoyl, phenyl, thienyl, furoyl and ethoxycarbonyl.

Suitable identities for R⁴ include benzenesulphonyl, 2-naphthalenesulphonyl, o-, m- or p-toluenesulphonyl, o-, m- or p-chlorobenzenesulphonyl, o-, m- or p-methoxy benzenesulphonyl, methanesulphonyl, dimethylaminosulphonyl, thienylsulphonyl, benzoyl, acetyl, furoyl and tert-butoxycarbonyl.

Suitable identities for R⁵ include hydroxy, methoxy, ethoxy, propoxy, benzyloxy, nitrile, fluorine, chlorine and bromine. Preferably, there is no more than one R⁵ substituent (i.e. p and q are 0 or 1), and when a single R⁵ substituent is present, it is preferably in the para-position relative to the indole nitrogen.

In the compounds of Formula I, p is preferably zero; R¹ and R² are preferably identical and represent hydrogen or methyl; R³ preferably represents hydrogen or benzoyl; and R⁴ preferably represents arylsulphonyl or dimethylaminosulphonyl. Examples of specific compounds in accordance with Formula I include:

2-[1-(benzenesulphonyl)-1H-indol-4-yl]ethylamine;

N,N-dimethyl 2-[1-(benzenesulphonyl)-1H-indol-4-yl]ethylamine;

N,N-dimethyl 3-[1-(benzenesulphonyl)-1H-indol-4-yl]propylamine; and

N,N-dimethyl 2-[1-(benzenesulphonyl)-2-benzoyl-1H-indol-4-yl]ethylamine.

In the compounds of Formula II, preferably R¹ and R² are identical and represent hydrogen or methyl, or together complete a pyrrolidinyl, piperidinyl, piperazinyl or 4-methylpiperazinyl ring; R³ preferably represents hydrogen or methyl; R⁴ preferably represents arylsulphonyl, thienylsulphonyl, benzoyl or tert-butoxycarbonyl; R⁵ preferably represents hydroxy, methoxy, benzyloxy or nitrile; and q is zero or 1. A sub-class of compounds in accordance with Formula II is defined by Formula II(a):

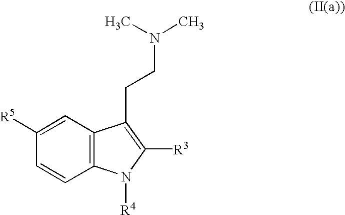

where R³, R⁴ and R⁵ have the same meanings as before.

Specific examples of compounds in accordance with Formula II(a) include:

N,N-dimethyl 2-[1-(benzenesulphonyl)-5-methoxy-1H-indol-3-yl]ethylamine;

N,N-dimethyl 2-[5-methoxy-1-(4-methyl benzenesulphonyl)-1H-indol-3-yl]ethylamine;

N,N-dimethyl 2-[1-(4-chlorobenzenesulphonyl)-5-methoxy-1H-indol-3yl]ethylamine;

N,N-dimethyl 2-[1-(3-chlorobenzenesulphonyl)-5-methoxy-1H-indol-3-yl]ethylamine;

N,N-dimethyl 2-[5-methoxy-1-(2-naphthalenesulphonyl)-1H-indol-3 -yl]ethylamine;

N,N-dimethyl 2-[5-methoxy-1-(4-methoxybenzenesulphonyl)-1H-indol-3-yl]ethylamine;

N,N-dimethyl 2-[1-(2-chlorobenzenesulphonyl)-5-methoxy-1H-indol-3-yl]ethylamine;

N,N-dimethyl 2-(1-benzoyl-5-methoxy-1H-indol-3-yl)ethylamine;

N,N-dimethyl 2-[5-methoxy-1-(2-thiophenesulphonyl)-1H-indol-3-yl]ethylamine;

N,N-dimethyl 2-[(1-benzenesulphonyl)-5-methoxy-2-methyl-1H-indol-3-yl]ethylamine;

N,N-dimethyl 2-(1-benzenesulphonyl-1H-indol-3-yl)ethylamine;

N,N-dimethyl 2-(1-methylsulphonyl-1H-indol-3-yl)ethylamine;

N,N-dimethyl 2-(5-methoxy-1-methylsulphonyl-1H-indol-3-yl)ethylamine;

3-(2-dimethylamino-ethyl)-5-hydroxy-1H-indole-1-carboxylic acid tert-butyl ester;

N,N-dimethyl 2-[(1-benzenesulphonyl)-5-benzyloxy-1H-indol-3-yl)]ethylamine;

N,N-dimethyl 2-[(1-benzenesulphonyl)-5-hydroxy-1H-indol-3-yl)]ethylamine; and

N,N-dimethyl 2-[(1-benzenesulphonyl)-5-cyano-1H-indol-3-yl)]ethylamine.

A further sub-class of compounds in accordance with Formula II is defined by Formula II(b):

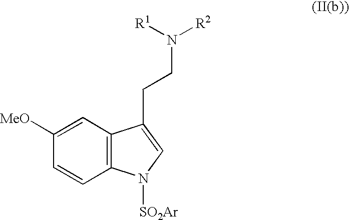

where R¹ and R² have the same meanings as before, and Ar represents an aryl or heteroaryl group.

Specific examples of compounds in accordance with Formula II(b) include:

2-[1-(benzenesulphonyl)-5-methoxy-1H-indol-3-yl]ethylamine;

1-benzenesulphonyl-5-methoxy-3-[(2-pyrrolidin-1-yl)ethyl]-1H-indole;

1-benzenesulphonyl-5-methoxy-3-[(2-piperidin-1-yl)ethyl]-1H-indole; and

1-benzenesulphonyl-5-methoxy-3-[(2-piperazin-1-yl)ethyl]-1H-indole.

A third sub-class of compounds in accordance with Formula II is defined by Formula II(c):

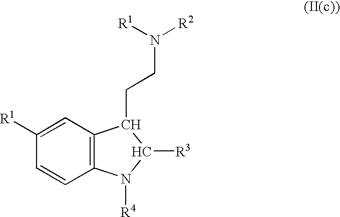

where R¹-R⁵ have the same meanings as before.

Specific examples of compounds in accordance with Formula II(c) include:

N,N-dimethyl 2-(1-benzenesulphonyl-5-methoxy-2,3-dihydro-1H-indol-3-yl)ethylamine.

In the compounds of Formula III, R¹ and R² are preferably identical and represent hydrogen or methyl; R³ preferably represents hydrogen; R⁴ preferably represents arylsulphonyl, especially p-toluenesulphonyl; R⁶ preferably represents hydroxy or methoxy; and p is preferably zero. Specific examples of compounds in accordance with Formula III include:

trans-4-dimethylamino-5-hydroxy-1-(4-methylbenzenesulphonyl)-1,3,4,5-tetrahydro-benz[c,d]indol-5-ol; and

trans-4-dimethylamino-5-methoxy-1-(4-methylbenzenesulphonyl)-1,3,4,5-tetrahydro-benz[c,d]indole.

The compounds of Formulae I-III, to be used according to the invention, can be prepared according to the methods described in U.S. Pat. No. 6,187,805 and GB 2,341,549.

The present invention relates to a method for the treatment or prophylaxis of obesity or for the reduction of food intake in mammals, including humans. The method comprises administering to a subject (e.g., a mammal, a human, a horse, a dog, or a cat) in need of such treatment a therapeutically effective amount of one or more compounds described above, or a composition having one or more compounds of formulae I-III in it.

The method delineated herein can also include the step of identifying that the subject is in need of treatment of the aforementioned diseases or conditions. The identification can be in the judgment of a subject or a health care professional and can be a subjective (e.g., opinion) or objective (e.g., measurable by a test or diagnostic method).

“An effective amount” refers to an amount of a compound that confers a therapeutic effect on the treated subject. The therapeutic effect may be objective (i.e., measurable by some test or marker) or subjective (i.e., subject gives an indication of or feels an effect). The dose level of the compounds described above, and the frequency of dosage of the specific combination, will vary depending on a variety of factors including the potency of each specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 150 mg per kilo of body weight, preferably from about 0.01 mg to about 100 mg per kilo of body weight, especially from about 0.1 to about 50 mg per kilo of body weight the compound of formula I, administered singly or multiply in doses, e.g. dosages of from about 0.01 mg to about 25 mg each. Usually, such a combined dosage is given orally but e.g. parenteral or rectal administration may also be chosen. A currently preferred oral daily dosage for a human subject is from about 1 to about 80 mg, preferably from about 1 to about 50 mg per day.

The compounds discussed above can be brought into suitable galenic forms, such as compositions for oral use, for injection, for nasal spray administration or the like, in accordance with accepted pharmaceutical procedures. Such pharmaceutical compositions according to the invention comprise an effective amount of one, or optionally more, compound(s) discussed above in association with compatible pharmaceutically acceptable carrier materials, or diluents, as are well known in the art. The carriers may be any inert material, organic or inorganic, suitable for oral, enteral, rectal, percutaneous, subcutaneous or parenteral administration, such as: water, gelatin, gum arabicum, lactose, microcrystalline cellulose, starch, sodium starch glycolate, calcium hydrogen phosphate, magnesium stearate, talcum, colloidal silicon dioxide, and the like. Such compositions may also contain other pharmacologically active agents, and conventional additives, such as stabilizers, wetting agents, emulsifiers, flavoring agents, buffers, and the like.

The compositions according to the invention can e.g. be made up in solid or liquid form for oral administration, such as tablets, pills, capsules, powders, syrups, elixirs, dispersible granules, cachets, suppositories and the like, in the form of sterile solutions, suspensions or emulsions for parenteral administration, sprays, e.g. a nasal spray, transdermal preparations, e.g. patches, and the like.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE

Effect on Food Intake of N,N-Dimethyl 2-[1-(benzenesulphonyl)-5-methoxy-1H-indol-3-yl]ethylamine by single dose subcutaneous administration in ob/ob mice.

Animals

Obese (ob/ob) mouse is selected as the primary animal model for screening as this mutant mouse consumes high amounts of food resulting in a high signal to noise ratio. To further substantiate and compare efficacy data, the effect of the compounds on food consumption is also studied in wild type (C57BL/6J) mice. The amount of food consumed during 15 hours of infusion of compounds is recorded.

Male mice (obese C57BL/6JBom-Lep^{ob} and lean wild-type C57B1/6JBom; Bomholtsgaard, Denmark) 8-9 weeks with an average body weight of 50 g (obese) and 25 g (lean) are used in all the studies. The animals are housed singly in cages at 23±1° C., 40-60% humidity and have free access to water and standard laboratory chow. The 12/12-h light/dark cycle is set to lights off at 5 p.m. The animals are conditioned for at least one week before start of study.

Compound

The test compound, N,N-Dimethyl 2-[1-(benzenesulphonyl)-5-methoxy-1H-indol-3-yl]ethylamine, was dissolved in 25% Polyetylenglykol 400 (PEG 400) plus 0.1% Tween 80. +sodium actetate till pH 5. Doses of 30, 50 and 50 mg kg⁻¹ were used. The purity of the test compounds is of analytical grade.

Animal dosage

| Sex, Strain and Species: | Male C57BL/6J-Lep^{ob}/Lep^{ob} |
|---|---|
| | (ob/ob) mouse |
| Age & Weight: | approx 10 weeks (˜45 gram) |
| Route: | sc |
| Dose (mg salt/kg): | 10, 30, 50 |
| Injection volume (ml) | 0.25 |
| Dose volume (ml/kg): | 5 |
| No of administrations: | Single dose |
| Time of administration: | 4.30 pm (lights off 5 pm) |
| No. of animals/ treatment group: | 8 |
| Total no. of animals: | 32 |

Experimental Design

The animals were divided into four groups containing 8 animals each and treated with vehicle plus three dosages of the test compound. Food consumption, total motor activity and water consumption were measured continuously for 22 h following start of recording in a computer-assisted operant test cage system (Eater meter). The animals were habituated for two days. The third day was defined as the day before treatment (basal). On the fourth day the animals were treated with test compound just before dark onset (5 pm) and data recorded cumulatively for 3 h, 6 h, 12 h and 21 h. Water consumption during 22 h was also measured by weighing the days before and after treatment.

Statistical Evaluation

Animals were randomized according to body weight and treatment assigned in a cage- and room-wise order. The food intake data are corrected for spillage during the test period of 22 h. Spillage at other time points were calculated proportionally to that of the 22 h spillage. The values are expressed as mean±SEM both as the change in gram from basal level and as percent of basal level. Statistical evaluation was performed on the percentage basal values using Kruskal-Wallis one-way ANOVA and, if significant, followed by Mann-Whitney U-test for test of significance between treatment groups. ID₂₀ values (mg salt/kg) are estimated by visual inspection and indicate the dose causing 20% inhibition of response.

Results

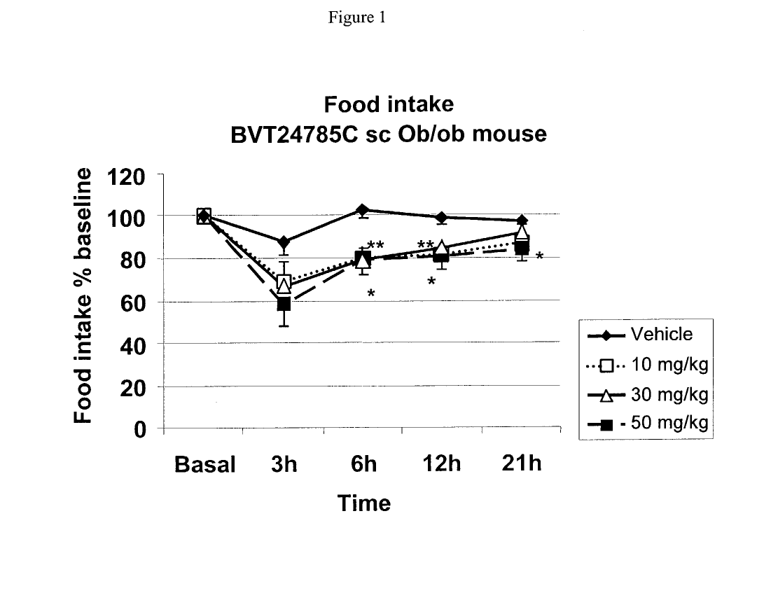

N,N-Dimethyl 2-[1-(benzenesulphonyl)-5-methoxy-1H-indol-3-yl]ethylamine decreases food intake significantly at 6 h and 12 h following administration by approximately 15-20%, see FIG. 1. This effect was not dose-dependent since all doses overlapped. Total activity was not affected

Approximate minimum effective dose 10 mg/kg (borderline statistical significance of 15-20% inhibition) for food intake.

What is claimed is:

1. A method for the treatment or prevention of obesity or for the reduction of food intake, comprising administering to a patient in need of such treatment an effective amount of a compound, or a pharmaceutically acceptable salt or prodrug thereof, having a structure in accordance with Formula I, II or III: 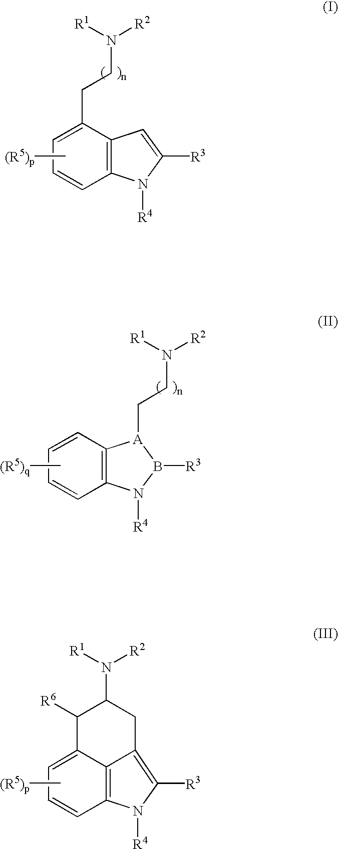
wherein
n is 1 or 2;
p is 0, 1, 2 or 3;
q is 0, 1, 2, 3 or 4;
R₁ and R² independently represent hydrogen, C₁₋₆ alkyl or aryl (C₁₋₆)alkyl, or together represent the atoms necessary to complete a heterocycloalkyl group comprising the nitrogen atom to which R¹ and R² are attached;
R³ represents hydrogen, C₁₋₆ alkyl, C₂₋₆ alkenyl, C₂₋₆ alkynyl, aryl(C₁₋₆)alkyl, aryl, heteroaryl, arylcarbonyl, heteroarylcarbonyl, C₁₋₆ alkylcarbonyl, or (C₁₋₆)alkoxycarbonyl;
R⁴ represents arylsulphonyl, heteroarylsulphonyl, C₁₋₆ alkylsulphonyl, di(C₁₋₆)alkylaminosulphonyl, arylcarbonyl, C₁₋₆ alkylcarbonyl, heteroarylcarbonyl or C₁₋₆ alkoxycarbonyl;
each R⁵ independently represents hydrogen, hydroxy, C₁₋₆ alkoxy, aryl(C₁₋₆)alkoxy, nitrile or halogen;
R¹⁻⁶ represents hydrogen, hydroxy or C₁₋₆ alkoxy; and
A—B represents C═C or CH—CH.

2. The method according to claim 1 in which said compound is in accordance with said Formula I or II or III wherein:
R¹ and R² independently represent hydrogen, methyl, ethyl, propyl or benzyl, or R¹ and R² in combination represents pyrrolidinyl, piperidinyl, piperazinyl, 4-methylpiperazinyl or morpholinyl;
R³ represents hydrogen, methyl, ethyl, benzyl, allyl, propargyl, benzoyl, phenyl, thienyl, furoyl, or ethoxycarbonyl;
R⁴ represents benzenesulphonyl, 2-naphthalenesulphonyl, o-, m- or p-toluenesulphonyl, o-, m- or p-chlorobenzenesulphonyl, o-, m- or p-methoxybenzenesulphonyl, methanesulphonyl, dimethylaminosulphonyl, thienylsulphonyl, benzoyl, acetyl, furoyl or tert-butoxycarbonyl; and
R⁵ represents hydrogen, hydroxy, methoxy, ethoxy, propoxy, benzyloxy, nitrile, fluorine, chlorine or bromine.

3. The method according to claim 1 in which the compound is selected from:
(a) compounds of Formula I in which p is zero; R¹ and R² are identical and represent hydrogen or methyl; R³ represents hydrogen or benzoyl; R⁴ represents arylsulphonyl or heteroarylsulphonyl; and R⁵ represent hydrogen or methoxy; and
(b) compounds of Formula II in which R¹ and R² are identical and represent hydrogen or methyl, or together complete a pyrrolidinyl, piperidinyl, piperazinyl or 4-methylpiperazinyl ring; R³ represents hydrogen or methyl; R⁴ represents arylsulphonyl, thienylsulphonyl, benzoyl or tert-butoxycarbonyl; R⁵ represents, hydroxy, methoxy, benzyloxy or nitrile; and q is zero or 1.
(c) compounds of Formula III in which R¹ and R² are identical and represent hydrogen or methyl; R³ represents hydrogen; R⁴ represents arylsulphonyl; R⁶ represents hydrogen, hydroxy or methoxy; and p is zero.

4. The method according to claim 1 in which the compound is in accordance with Formula I.

5. The method according to claim 4, wherein the compound is:
2-[1-(benzenesulphonyl)-1H-indol-4-yl]ethylamine;
N,N-dimethyl 2-[1-(benzenesulphonyl)-1H-indol-4-yl]ethylamine;
N,N-dimethyl 3-[1-(benzenesulphonyl)-1H-indol-4-yl]propylamine; or
N,N-dimethyl 2-[1-(benzenesulphonyl)-2-benzoyl-1H-indol-4-yl]ethylamine.

6. The method according to claim 1 in which the compound is in accordance with Formula II(a): 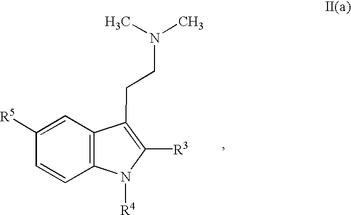
wherein R³, R⁴, and R⁵ are as defined in claim 1.

7. The method according to claim 6 in which the compound is:
N,N-dimethyl 2-[1-(benzenesulphonyl)-5-methoxy-1H-indol-3-yl]ethylamine;
N,N-dimethyl 2-[5-methoxy-1-(4-methyl benzenesulphonyl)-1H-indol-3-yl]ethylamine;
N,N-dimethyl 2-[1-(4-chlorobenzenesulphonyl)-5-methoxy-1H-indol-3yl]ethylamine;
N,N-dimethyl 2-[1-(3-chlorobenzenesulphonyl)-5-methoxy-1H-indol-3-yl]ethylamine;
N,N-dimethyl 2-[5-methoxy-1-(2-naphthalenesulphonyl)-1H-indol-3-yl]ethylamine;
N,N-dimethyl 2-[5-methoxy-1-(4-methoxybenzenesulphonyl)-1H-indol-3-yl]ethylamine;
N,N-dimethyl 2-[1-(2-chlorobenzenesulphonyl)-5-methoxy-1H-indol-3-yl]ethylamine;
N,N-dimethyl 2-(1-benzoyl-5-methoxy-1H-indol-3-yl)ethylamine;
N,N-dimethyl 2-[5-methoxy-1-(2-thiophenesulphonyl)-1H-indol-3-yl]ethylamine;
N,N-dimethyl 2-[(1-benzenesulphonyl)-5-methoxy-2-methyl-1H-indol-3-yl]ethylamine;
N,N-dimethyl 2-(1-benzenesulphonyl-1H-indol-3-yl)ethylamine;
N,N-dimethyl 2-(1-methylsulphonyl-1H-indol-3-yl)ethylamine;
N,N-dimethyl 2-(5-methoxy-1-methylsulphonyl-1H-indol-3-yl)ethylamine;
3-(2-dimethylamino-ethyl)-5-hydroxy-1H-indole-1-carboxylic acid tert-butyl ester;
N,N-dimethyl 2-[(1-benzenesulphonyl)-5-benzyloxy-1H-indol-3-yl)]ethylamine;
N,N-dimethyl 2-[(1-benzenesulphonyl)-5-hydroxy-1H-indol-3-yl)]ethylamine; or
N,N-dimethyl 2-[(1-benzenesulphonyl)-5-cyano-1H-indol-3-yl)]ethylamine.

8. The method according to claim 1 in which the compound is in accordance with Formula II(b): 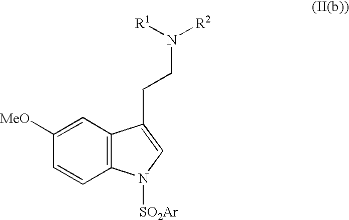
wherein R¹ and R² are as defined in claim 1, and Ar represents an aryl or heteroaryl group.

9. The method according to claim 8, wherein R¹ and R² are methyl groups.

10. The method according to claim 8, wherein Ar is selected from the group consisting of phenyl, 2-thienyl, and 3-chlorophenyl.

11. The method according to claim 8, wherein the compound is:
2-[1-(benzenesulphonyl)-5-methoxy-1H-indol-3-yl]ethylamine;
1-benzenesulphonyl-5-methoxy-3-[(2-pyrrolidin-1-yl)ethyl]-1H-indole;
1-benzenesulphonyl-5-methoxy-3-[(2-piperidin-1-yl)ethyl]-1H-indole; or
1-benzenesulphonyl-5-methoxy-3-[(2-piperazin-1-yl)ethyl]-1H-indole.

12. The method according to claim 1 in which the compound is in accordance with Formula II(c): 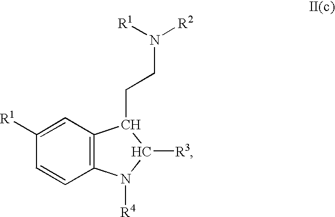
wherein R¹, R², R³, R⁴, and R⁵ are as defined in claim 1.

13. The method according to claim 12, wherein the compound is
N,N-dimethyl 2-(1-benzenesulphonyl-5-methoxy-2,3-dihydro-1H-indol-3-yl)ethylamine.

14. The method according to claim 1 in which the compound is in accordance with Formula III.

15. The method according to claim 14, wherein the compound is
trans-4-dimethylamino-5-hydroxy-1-(4-methylbenzenesulphonyl)-1,3,4,5-tetrahydro-benz[c,d]indol-5-ol; or
trans-4-dimethylamino-5-methoxy-1-(4-methylbenzenesulphonyl)-1,3,4,5-tetrahydro-benz[c,d]indole.

16. The method according to claim 1, wherein the said compound is N,N-dimethyl-2-[1-(benzenesulphonyl)-5-methoxy-1H-indol-3-yl]ethylamine.
